(19) United States
(12) Patent Application Publication
(10) Pub. No.: US 2024/0261352 A1
(43) Pub. Date: Aug. 8, 2024

(54) PRE-EVAPORATION STANDARDIZATION OF EXTRACTED PSYCHOACTIVE COMPOUNDS

(71) Applicant: PSILO SCIENTIFIC LTD, Vancouver (CA)

(72) Inventors: Ryan MOSS, Vancouver (CA); Benjamin LIGHTBURN, Vancouver (CA); Lisa RANKEN, Lake Country (CA)

(21) Appl. No.: 18/681,683

(22) PCT Filed: Jul. 25, 2022
(86) PCT No.: PCT/CA2022/051146

§ 371 Date: Feb. 6, 2024

(30) Foreign Application Priority Data
Aug. 6, 2021 (CA) 3127180

(51) Int. Cl. A61K 36/07; A61K 31/4045; A61K 31/675; A61K 47/02; A61K 47/22; A61K 47/36

(52) U.S. Cl. CPC A61K 36/078; A61K 31/4045; A61K 31/675; A61K 47/02; A61K 47/22; A61K 47/36; A61K 2236/331; A61K 2236/333; A61K 2236/51; A61K 2236/53

(57) ABSTRACT

This invention relates to the extraction of psychoactive compounds from psychedelic organisms for use in medicine. Raw psychedelic organisms are dried and pulverized. The psychoactive compounds are extracted using a solvent to result in an extraction slurry. The extraction slurry is filtered and pH-adjusted if necessary. The filtrate is then standardized to provide, when dried, a known concentration of the psychoactive alkaloids that have been extracted. The solvent is then evaporated from the filtrate to form the extract. The extract may be mixed with water and spray dried. The standardization process is run before or after full or partial evaporation of the solvent. The result is a powdered extract with a precisely defined concentration of psychoactive compounds.

### TECHNICAL FIELD

This application relates to the extraction of active ingredients from organisms. More specifically, it relates to extracting psychoactive compounds from psychedelic organisms and forming an extract of known concentration.

### BACKGROUND

Varieties of mushrooms have played important roles in most societies. The active ingredients in mushrooms, especially psilocybin mushrooms with psychoactive compounds, such as psilocybin, psilocin, baeocystin, norbaeocystin, ibotenic acid, and norpsilocin, have been found to have medicinal properties including relief of symptoms of various diseases and conditions. The concentration of active psilocybin mushroom compounds varies not only from species to species, but also from mushroom to mushroom within a given species, subspecies or variety. The same holds true even for different parts of the same mushroom or mycelium.

Various methods of extraction, which have been used to separate natural extracts from a variety of mushrooms, have resulted in difficulties with large crop-to-crop variability. This is as well as the problem of a large variability within a single plant or fungus in terms of the concentration of the active psychoactive compound and its stability. Different solvent choices extract the psychoactive compounds equally, some of them selectively extract one or the other, and some convert the compounds between each other or degrade them into non-psychoactive compounds. Many extraction processes for extracting standardized concentrations of the compounds for direct medical use are usually complex. This results in expensive extraction processes and a high cost of isolated, natural extracts.

U.S. Pat. No. 3,183,172 to Heim et al. relates to an industrial process for the isolation of active compounds from mushrooms grown under predetermined conditions. With the predetermined growing conditions, mushrooms grow with ten times more active mycelium and sclerotium, and increased concentrations of psychoactive compounds. However, a large portion of the target compounds are lost during the extraction process or not extracted at all. This problem is significant with respect to very potent extracts of psilocybin mushrooms, considering that a normal dose for use ranges from only 5 mg to 25 mg. The extracted psychoactive compounds are generally without a stable and standardized concentration.

To date, the focus has largely been on synthetic preparations of these compounds because of the many difficulties associated with naturally extracted preparations. It is currently infeasible and expensive to extract psilocybin from mushrooms, and even the best chemical synthesis methods require expensive and difficult-to-source starting substrates.

Accordingly, there is a need of methods to produce high efficiency, standardized preparations of the target compounds for medical use while using acceptable solvent systems to create a more consistent supply chain.

This background information is provided to reveal information believed by the applicant to be of possible relevance to the present invention. No admission is necessarily intended, nor should be construed, that any of the preceding information constitutes prior art against the present invention.

### SUMMARY OF INVENTION

The present invention is directed to an extraction process of psychoactive compounds from psychedelic organisms, for example, the Psilocybe cubensis species of psychedelic mushroom. The principal psychoactive compounds in Psilocybe cubensis include psilocybin and psilocin. In particular, the extraction process of psychoactive compounds involves drying fresh Psilocybe cubensis, followed by grinding or pulverizing, extraction with a solvent in one or more steps, one or more steps of filtration, optional adjustment of the pH if the solvent is acidic (acid/water/alcohol) or alkaline (base/water/alcohol), evaporation of the solvent, and standardization before evaporation of the solvent or after the evaporation of some or all of the solvent. Optionally, the process includes drying to result in a final powdered psilocybin mushroom extract.

This summary does not necessarily describe all features of the invention.

Disclosed is a process for forming an extract with a specified concentration of psychoactive alkaloids from a dried, raw psychedelic organism comprising the steps of: soaking a biomass of dried, raw psychedelic organism with a solvent consisting of one or more members selected from the group consisting of C1-C4 aliphatic alcohols, C3-C4 ketones, water, a buffered acid and a buffered alkali in order to dissolve the psychoactive alkaloids in the solvent; filtering an undissolved portion of the biomass from the solvent to result in a filtrate; measuring a psychoactive alkaloid content in the filtrate; measuring a dry mass content in the filtrate; using the psychoactive alkaloid content, the dry mass content and the specified concentration to determine a quantity of excipient to add to the filtrate in order to obtain the specified concentration of the psychoactive alkaloids in the extract; standardizing the filtrate by adding thereto the quantity of excipient; and evaporating all or some of the solvent from the filtrate to leave the extract or a residue comprising the extract. The process may include mixing the extract or the residue in water to form a mixture, and spray-drying the mixture to result in a powdered form of the extract.

### BRIEF DESCRIPTION OF DRAWINGS

The following drawings illustrate embodiments of the invention, which should not be construed as restricting the scope of the invention in any way.

FIG. 1 is a high-level flowchart showing the key steps of a process for extracting psychoactive alkaloids from psilocybin fungus, according to an embodiment of the present invention.

FIG. 2 is a high-level flowchart showing the key steps of a process for extracting psychoactive alkaloids from psilocybin fungus, according to another embodiment of the present invention.

FIG. 3 is a flowchart showing more detailed steps of a process for extracting psychoactive alkaloids from Psilocybe cubensis using a 75% ethanol solvent, according to another embodiment of the present invention.

FIG. 4 is a flowchart showing more detailed steps of a process for extracting psychoactive alkaloids from Psilocybe cubensis using a hydro-ethanol solvent, according to another embodiment of the present invention.

FIG. 5 is a flowchart showing more detailed steps of a process for extracting psychoactive alkaloids from Psilocybe cubensis using a water solvent, according to another embodiment of the present invention.

FIG. 6 is a flowchart showing more detailed steps of a process for extracting psychoactive alkaloids from Psilocybe cyanescens using a methanol solvent, according to another embodiment of the present invention.

FIG. 7 is a flowchart showing more detailed steps of a process for extracting psychoactive alkaloids from Psilocybe cubensis using a buffered acidic solvent, according to another embodiment of the present invention.

FIG. 8 is a flowchart showing more detailed steps of a process for extracting psychoactive alkaloids Psilocybe cubensis using a buffered alkaline solvent, according to another embodiment of the present invention.

FIG. 9 is a flowchart showing more detailed steps of a process for extracting psychoactive alkaloids Psilocybe cubensis using methanol, according to another embodiment of the present invention.

FIG. 10 is a schematic diagram of the apparatus used for the extraction of psychoactive compounds according to embodiments of the present invention.

### DESCRIPTION

### A. Glossary

Psychedelic fungi, psilocybin fungi, or psilocybin mushrooms—these are a group of fungi that contain at least one psychoactive alkaloid, and generally contain psilocybin and psilocin. They may also contain other psychoactive alkaloids such as baeocystin, norbaeocystin, ibotenic acid and norpsilocin. The genera of these mushrooms include Copelandia, Gymnopilus, Inocybe, Panaeolus, Pholiotina, Pluteus, Amanita and Psilocybe. Psilocybe mushrooms—these form a genus of gilled mushrooms in the family Hymenogastraceae. Most species contain the psychedelic alkaloids psilocybin, psilocin and baeocystin.

Psilocybin—this is a psychedelic prodrug produced by numerous species of mushrooms, collectively known as psilocybin mushrooms. Psilocybin is converted by the body to psilocin, which has mind-altering effects such as euphoria and hallucinations, but can also lead to nausea and panic attacks.

Psychoactive alkaloid—this refers to alkaloids that upon ingestion are capable of changing brain function, resulting in alterations in perception, mood, consciousness, cognition or behavior, for example. Psychoactive alkaloids are abundant in nature and can be obtained from psychedelic organism sources such as a fungus, an animal, a mycelium, a spore, a plant, a bacterium, or a yeast. Examples of psychoactive alkaloids include, but are not limited to, psilocybin, psilocin, baeocystin, norbaeocystin, norpsilocin, aeruginascin, bufotenin, bufotenidine, 5-MeO-DMT (5-methoxy-N,N-dimethyltryptamine), N,N-dimethyltryptamine (DMT), ergine (LSA), ergonovine, ergometrine, ibotenic acid, muscimol, lysergic acid hydroxyethylamide (LSH), elymoclavine, ergometrinine, and/or chanoclavine.

The term “excipient” means any component added to an active ingredient to make a composition. An excipient is inert in relation to the active ingredient, in that it essentially does not act in the same way as the active ingredient. An excipient may be completely inert, or it may have some other property that protects the integrity of the active ingredient or assists its uptake into the human body. There are multiple types of excipient, each having a different purpose, and a given excipient may fulfill more than one purpose. Examples of types of excipient include flowability agents, flavorants, colorants, palatants, antioxidants, bioavailability-increasing agents, viscosity modifying agents, tonicity agents, drug carriers, sustained-release agents, comfort-enhancing agents, emulsifiers, solubilizing aids, lubricants, binding agents and stabilizing agents. Specific excipients include pectin, rice husks, rice, xanthum gum, gum arabic, beta cyclodextrin, alpha cyclodextrin, microcrystalline cellulose, sorbitol, dextrose, guar gum, acacia gum, cellulose gum, talc, magnesium stearate.

The term “carrier” means an excipient that aids in delivery of the active ingredient or provides bulk to the composition. The amount of carrier included in a composition can vary widely in order to control the concentration of the active ingredient in the composition. An example of a carrier is starch, maltodextrin, tapioca maltodextrin or rice maltodextrin, alpha and beta cyclodextrin, microcrystalline cellulose (MCC), gum arabic, xanthum gum, guar gum, or cellulose gum.

### B1. Primary General Process

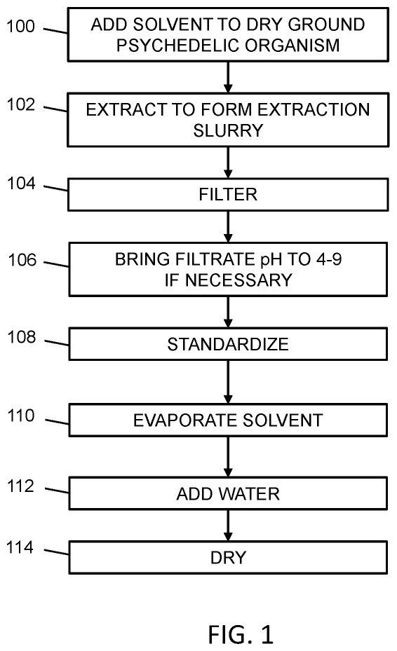

Referring to FIG. 1, a flowchart is shown of the basic steps of the extraction process for extracting psychoactive compounds from psychedelic organisms. In step 100, a solvent is added to a biomass of one or more dried and ground raw organisms. The raw organisms include, for example, Psilocybe cubensis mushrooms, Psilocybe cyanescens mushrooms, Amanita muscaria mushrooms or a mixture of any of these. Other species of psychedelic mushrooms or psychedelic organisms may also be used.

The parts of the mushrooms, if used, include, for example, caps, gills, stems, and hyphae, and more particularly, any part of the psilocybin mushroom or mycelium can be included. In other cases, the raw psilocybin fungus parts used include only caps, or only stems, or only gills, or only hyphae or only mycelium or any mixture thereof. In still other cases, parts of the raw psilocybin fungus used are those that would normally be considered waste, in which valuable psychoactive compounds are found only in lower concentrations. The mushroom parts may be ground using a milling machine or pulverization device, for example.

Ideally, the moisture content of the raw psychedelic organism after drying is low compared to the total dried biomass weight. For example, the moisture content may be under 5% for smaller scale extractions and under 10% for larger scale extractions. Wet mushrooms, e.g. with a moisture above 80%, will degrade rapidly. Dried biomass lends itself well to extraction since the drying process usually breaks down cell walls, allowing solvent to capture the molecules inside. The temperature of the oven and the drying time depend on how much moisture is in the raw psychedelic organism, and on the quantity of raw psychedelic organism.

The solvent may be selected from a range of different solvents, including lower aliphatic alcohols (C=1, 2, 3 or 4), C3-C4 ketones, water, alcohol-water mixtures, C3-C4 ketone-water mixtures, lower aliphatic alcohol and C3-C4 ketone mixtures, buffered alcohol-water mixtures, buffered C3-C4 ketone-water mixtures, strong alkaline buffers, strong alkali buffered lower aliphatic alcohols, strong alkali buffered C3-C4 ketones, strong acidic buffers, strong acid buffered lower aliphatic alcohols and strong acid buffered C3-C4 ketones. A wide range of solvent to solid ratios can be used. Typically, a 1 to 50:1 solvent-solid ratio (L:kg) may be used for the extraction. The amount of solvent used generally varies according to the weight of the raw psychedelic organism.

In step 102, as a result of adding the solvent, and soaking the biomass of dried, raw psychedelic organism in the solvent, essential elements or psychoactive alkaloids found in the biomass dissolve into the solvent. The solvent may be at a low or high temperature, and pressure may be applied to the solvent. In some embodiments the solvent is at room temperature. The optimal temperature of extraction varies depending on the solvent type used for the process. However, the optimal temperature for extraction is in range of 5-95° C. The useful temperature range spans most of the liquid state of the solvent used, and upper and lower limits are determined by physical practicalities and limits of the available apparatus. Still, the temperature of the solvent may be outside of this range in other embodiments. The duration of the extraction is from 10 minutes to 12 hours, with or without agitation. Optimum duration is determined by experimentation, and depends on the chosen solvent and the strength of agitation in the extraction vessel.

If pressure is applied it may be in the range of 50 kPa-100 MPa above atmospheric (7-15000 psig). The lower limit of pressure is indicative of when a benefit is seen in the rate at which the psychoactive alkaloids dissolve in the solvent, since the increased pressure may increase the reaction kinetics of the dissolution of the psychoactive alkaloids into the solvent. The upper limit is determined by what is physically practical given the constraints of equipment to safely operate under high pressure. Nevertheless, other pressures may be used. Solvent composition, particle size and the temperature of extraction will determine how much pressure needs to be applied.

The extraction results in an extraction slurry, which is formed of undissolved and insoluble solids from the biomass, and solvent, which now carries dissolved extract. Some of the undissolved solids may be undesirable components.

In step 104, the extraction slurry is filtered, resulting in a residue (i.e. the undissolved portion of the biomass) and filtrate. The filtering step may be carried out with the extraction slurry still hot, or it may first be allowed to cool. The extraction and filtration steps may be repeated multiple times on the same residue, with a fresh batch of solvent, which may have the same composition as the first solvent or it may be a different solvent.

In step 106, if the filtrate results from using a strongly acidic or alkaline solvent, then the filtrate is brought closer to neutral, e.g. to a pH between 4 and 9 or thereabouts. Desirable effects, such as more complete extraction, or preservation of the alkaloids from decomposition, or the ability to selectively extract certain specific alkaloids, are seen during the extraction stage when stronger acids or alkalis are used compared to weaker ones.

In step 108, standardization of the filtrate takes place. The aim is to stabilize the extract by adding sufficient stabilizer (e.g. ascorbic acid and silica), and then titrating with a carrier such as maltodextrin to result in a final, known concentration of psychoactive alkaloids. The filtrate is analyzed for dry mass concentration and alkaloid content. The liquid component of the filtrate is first analyzed using a loss-on-drying analysis and high performance liquid chromatography coupled with diode array detection or mass spectrometry to determine the alkaloid content. Depending on the determined alkaloid content, non-toxic excipients are added to the filtrate so as to provide a desired ratio between the weight of alkaloid and total weight of excipient in the filtrate. The added carriers, blending agents, flow aids, other excipients etc. that may be used include maltodextrin from corn, potato or tapioca for example, gum arabic, silicon dioxide, microcrystalline cellulose, ascorbic acid, sodium benzoate, sodium phosphate, sodium citrate, rice hulls, and rice. A combination of any of these excipients may be used.

In step 110, evaporation of the solvent or substantially all of the solvent from the filtrate results in solids or a slurry. If the solvent is methanol, then all of it is evaporated to reduce the likelihood of toxicity. For other solvents, there may be some residual solvent. In some cases, an amount of solvent may be purposely left unevaporated.

Water is then added to the solids or slurry in step 112. The solids tend not to dissolve back into solution because they are less soluble in water than in methanol and ethanol, for example. Also, the solids may be less soluble in the colder water that is added back than the warmer or hotter water that may have been used for the extraction. Another reason is saturation of the solution, or that some of the solids are irreversibly precipitated.

In step 114, the resulting water-based slurry is dried to remove the remaining solvent, if any, and the water, resulting in a powdered psychedelic organism extract with a known concentration by weight of psychoactive compound(s). The extract is a powdered extract that may have, for example, a total psychoactive alkaloid concentration of 0.1-10% by dry weight. Other compounds may be included in the extract. These may be sugars, proteins, carbohydrates and fats, and may make up about half of the extract. Step 114 is optional, as it may be the intention to produce a liquid extract instead of a powdered extract.

### B2. Secondary General Process

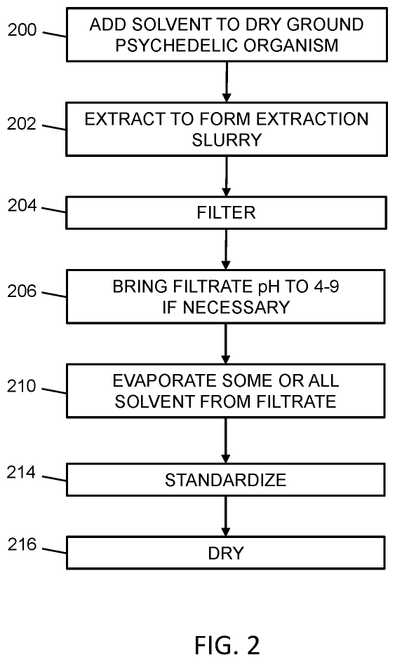

Referring to FIG. 2, a secondary process differs from the primary process in such a way that the standardization step 214 is carried out after the evaporation of some or all the solvent from the filtrate in step 210. In step 200, a solvent is added to a biomass of dried and ground, raw psychedelic organism. The raw psychedelic organism may be a psilocybin fungus, for example, that includes Psilocybe cubensis mushrooms, Psilocybe cyanescens mushrooms, Amanita muscaria mushrooms or a mixture of any of these. Other species of psychedelic mushrooms may also be used.

The parts of the mushrooms, if used, include, for example, caps, gills, stems, and hyphae, and more particularly, any part of the psilocybin mushroom or mycelium can be included. In other cases, the raw psilocybin fungus parts used include only caps, or only stems, or only gills, or only hyphae or only mycelium or any mixture thereof. In still other cases, parts of the raw psilocybin fungus used are those that would normally be considered waste, in which valuable psychoactive compounds are found only in lower concentrations. The mushroom parts may be ground using a milling machine or pulverization device, for example.

Ideally, the moisture content of the raw psychedelic organism (e.g. plant material) after drying is low compared to the total dried biomass weight. For example, the moisture content may be under 5% for smaller scale extractions and under 10% for larger scale extractions. Wet mushrooms, e.g. with a moisture above 80%, will degrade rapidly. Dried biomass lends itself well to extraction since the drying process usually breaks down cell walls, allowing solvent to capture the molecules inside. The temperature of the oven and the drying time depend on how much moisture is in the raw psychedelic organism, and on the quantity of raw psychedelic organism.

The solvent may be selected from a range of different solvents, including lower aliphatic alcohols (C=1, 2, 3 or 4), C3-C4 ketones, water, alcohol-water mixtures, C3-C4 ketone-water mixtures, lower aliphatic alcohol and C3-C4 ketone mixtures, buffered alcohol-water mixtures, buffered C3-C4 ketone-water mixtures, strong alkaline buffers, strong alkali buffered lower aliphatic alcohols, strong alkali buffered C3-C4 ketones, strong acidic buffers, strong acid buffered lower aliphatic alcohols and strong acid buffered C3-C4 ketones. A wide range of solvent to solid ratios can be used. Typically, a 1 to 50:1 solvent-solid ratio (L:kg) may be used for the extraction. The amount of solvent used generally varies according to the weight of the raw psychedelic organism.

In step 202, as a result of adding the solvent, and soaking the biomass of dried, raw psychedelic organism in the solvent, essential elements or psychoactive alkaloids found in the biomass dissolve into the solvent. The solvent may be at a low or high temperature, and pressure may be applied to the solvent. In some embodiments the solvent is at room temperature. The optimal temperature of extraction varies depending on the solvent type used for the process. However, the optimal temperature for extraction is in range of 5-95° C. The useful temperature range spans most of the liquid state of the solvent used, and upper and lower limits are determined by physical practicalities and limits of the available apparatus. Still, the temperature of the solvent may be outside of this range in other embodiments. The duration of the extraction is from 10 minutes to 12 hours, with or without agitation. Optimum duration is determined by experimentation, and depends on the chosen solvent and the strength of agitation in the extraction vessel.

If pressure is applied it may be in the range of 50 kPa-100 MPa above atmospheric (7-15000 psig). The lower limit of pressure is indicative of when a benefit is seen in the rate at which the psychoactive alkaloids dissolve in the solvent, since the increased pressure may increase the reaction kinetics of the dissolution of the psychoactive alkaloids into the solvent. The upper limit is determined by what is physically practical given the constraints of equipment to safely operate under high pressure. Nevertheless, other pressures may be used. Solvent composition, particle size and the temperature of extraction will determine how much pressure needs to be applied.

The extraction results in an extraction slurry, which is formed of undissolved and insoluble solids from the biomass, and solvent, which now carries dissolved extract. Some of the undissolved solids may be undesirable components.

In step 204, the extraction slurry is filtered, resulting in a residue (i.e. the undissolved portion of the biomass) and filtrate. The filtering step may be carried out with the extraction slurry still hot, or it may first be allowed to cool. The extraction and filtration steps may be repeated multiple times on the same residue, with a fresh batch of solvent, which may have the same composition as the first solvent or it may be a different solvent.

In step 206, if the filtrate results from using a strongly acidic or alkaline solvent, then the filtrate is brought closer to neutral, e.g. to a pH between 4 and 9 or thereabouts. Desirable effects, such as more complete extraction, or preservation of the alkaloids from decomposition, or the ability to selectively extract certain specific alkaloids, are seen during the extraction stage when stronger acids or alkalis are used compared to weaker ones.

In step 210, evaporation of some or all of the solvent from the filtrate results in a concentrated slurry (liquid and solids) or just solids. If the solvent is methanol, then all of it is evaporated to reduce the likelihood of toxicity. For other solvents, only some of the solvent needs to be evaporated. In the case where solids are obtained from the evaporation, water is added to the solids to form a concentrated slurry. The solids tend not to dissolve back into solution because they are less soluble in water than in methanol and ethanol, for example. Also, the solids may be less soluble in the colder water that is added back than the warmer or hotter water that may have been used for the extraction. Another reason is saturation of the solution, or that some of the solids are irreversibly precipitated.

In step 214, standardization of the concentrated slurry takes place. The aim is to stabilize the extract by adding sufficient stabilizer (e.g. ascorbic acid and silica), and then titrating with a carrier such as maltodextrin to result in a final, known concentration of psychoactive alkaloids. The slurry is analyzed for dry mass concentration and alkaloid content. The liquid component of the concentrated slurry is first analyzed using a loss-on-drying analysis and high performance liquid chromatography coupled with diode array detection or mass spectrometry to determine the alkaloid content. Depending on the determined alkaloid content, non-toxic excipients are added to the concentrated slurry so as to provide a desired ratio between the weight of alkaloid and total weight of excipient in the concentrated slurry. The added carriers, blending agents, flow aids, other excipients etc. that may be used include maltodextrin from corn, potato or tapioca for example, gum arabic, silicon dioxide, microcrystalline cellulose, ascorbic acid, sodium benzoate, sodium phosphate, sodium citrate, rice hulls, and rice. A combination of any of these excipients may be used.

In step 216, the concentrated slurry is dried to remove the remaining solvent or water, resulting in a powdered psychedelic organism extract with a known concentration by weight of psychoactive compound(s). The extract is a powdered extract that may have, for example, a total psychoactive alkaloid concentration of 0.1-10% by dry weight. Other compounds may be included in the extract. These may be sugars, proteins, carbohydrates and fats, and may make up about half of the extract. Step 216 is optional, as it may be the intention to produce a liquid extract instead of a powdered extract.

### C. Exemplary Embodiments

### 75% Ethanol Solvent

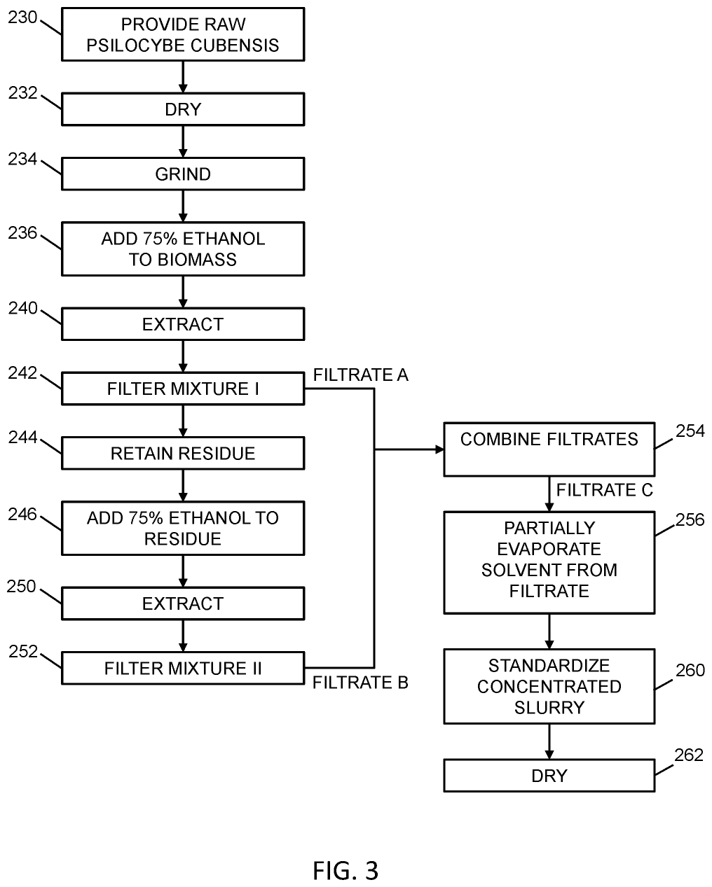

Referring to FIG. 3, an exemplary detailed process is shown for the extraction of psychoactive compounds from Psilocybe cubensis mushrooms using a 75% ethanol solvent.

In step 230, 2.5 kg of raw psilocybin mushrooms from the Psilocybe cubensis species is provided. In step 232, the raw psilocybin mushrooms are dried in a forced air oven at 25° C., for 10 hours. The aim is to dry the mushrooms so as not to significantly reduce their psychoactive alkaloid concentration. For example, if too high a temperature or too long a time at a specific temperature were used, the alkaloids may start to decompose. The resulting, dried biomass is 140 g. In step 234, the dried biomass is ground using a hammer mill or the equivalent, to a particle size of 200 mesh.

In step 236, a 5 kg quantity of the 75% (by weight) ethanol solvent, formed by mixing 3 parts of ethanol to 1 part of water by weight, is placed in an extraction vessel. The dried, ground biomass is also placed in the extraction vessel, which is heat-controlled and agitated.

The extraction proceeds in step 240 as the biomass soaks in the solvent. The temperature of the extraction process is 70° C., and the duration of extraction is 4 hours. The temperature remains constant during the extraction process.

In step 242, the resulting mixture of biomass solids and solvent with dissolved extract, is filtered while still hot, i.e. still at 70° ° C., or slightly lower due to ambient cooling. This removes a residue with undissolved psilocybin mushroom components from the filtrate. The filter used is a 10 μm sieve. The filtrate from this step is filtrate A. In step 244, the residue is retained and placed back into the extraction vessel. In step 246, another 5 kg of 75% ethanol is added to the retained residue.

In step 250, the extraction process of the residue continues at the same temperature as for the initial extraction step, i.e. at 70° C., for a time of 4 hours. Again, the temperature remains constant during the extraction process.

In step 252, the second resulting mixture, of biomass solids and solvent with dissolved extract, is filtered to remove the residue of unwanted solid material. The filter used is a 10 μm sieve. Note that in other embodiments a differently sized filter may be used here or in the prior filtration step, or the liquid may be decanted from the residue without filtering. In some embodiments, a centrifuge may be used to help separate the liquid from the residue. Filtrate B from the second filtration process may have a lower concentration of psychoactive compounds than filtrate A from the first filtration step. Filtrates A and B are then mixed in step 254 to result in bulk filtrate C. More extract can be obtained by splitting the solvent into two or more batches and using each one sequentially to soak the biomass, compared to using a single volume of solvent.

The bulk filtrate C is then processed with a rotary evaporator in step 256 to remove solvent until the volume of filtrate C is 2.5 L. At this point, the reduced amount of filtrate C is a concentrated slurry, due to the precipitation of water-insoluble components, for example.

The volume of 2.5 L is chosen because the mixture now has a low enough ethanol content that the excipients can be mixed in. By preferentially removing ethanol over water, which occurs naturally during the evaporation, it also gives the later spray-drying step a lower risk of explosion compared to if a 75% ethanol slurry were sprayed directly.

In step 260, after some of the solvent has been removed using the rotary evaporator, the concentrated slurry is then standardized. The standardization process uses a titration procedure to determine the concentration of the psychoactive alkaloids in the concentrated slurry. The standardization procedure entails adjusting the concentration of psychoactive alkaloids in the concentrated slurry so that when it is dried it achieves a desired target concentration, such as 1.00% by dry weight of psychoactive alkaloids. In this example, 4.7 g of ascorbic acid, 1.9 g of SiO₂ and 47 g of maltodextrin are added to the concentrated slurry.

In step 262, after the standardization process, the standardized concentrated slurry is dried using a bench-top spray dryer. This results in 100 g of powdered psilocybin mushroom extract with a total alkaloid concentration of 1.00% by weight. As can be seen, the purity of the extract can be defined as a percentage to a precision of two decimal places.

### 0-100% Ethanol Solvent

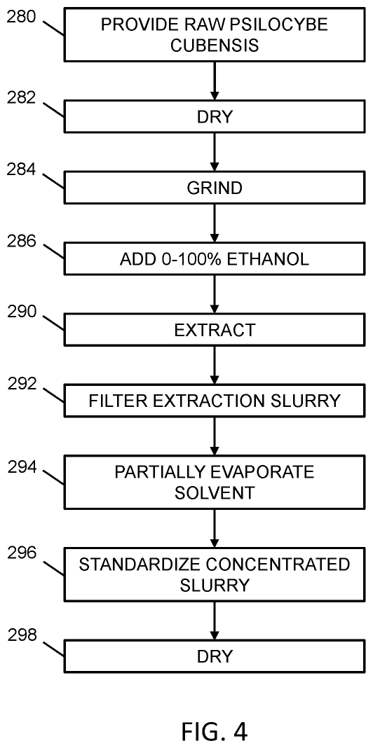

Referring to FIG. 4, a process is shown for the extraction of psychoactive compounds from Psilocybe cubensis using a general hydro-ethanol solvent. The solvent may range from a percentage of <1% of ethanol in water to 100% ethanol.

In step 280, 2.5 kg of raw psilocybin mushrooms from the Psilocybe cubensis species is provided. In step 282, the raw Psilocybe cubensis is dried in a forced air oven at 25° C. for 10 hours. In step 284, the resulting dried biomass is ground in a hammer mill or the equivalent, to particle size of 200 mesh.

In step 286, 5 kg of solvent, having a 0-100% ethanol concentration is added to an extraction vessel into which the ground biomass is placed. The extraction vessel is an agitated, heat-controlled vessel.

In step 290, the extraction proceeds as the biomass is soaked. The temperature of the extraction is elevated above room temperature to 70° C. Temperature and pressure, if applied, are generally selected so that the solvent does not boil if elevated temperatures are used. The duration of the extraction is 4 hours.

In the step 292, the extraction slurry is filtered to remove residue with undissolved Psilocybe cubensis from the filtrate. The residue may be treated with another extraction step if desired, and if so, the filtrate from the subsequent step is combined with the filtrate from the first filtration.

In step 294, solvent from the filtrate is partially evaporated using a rotary evaporator. The resulting concentrated slurry is then subjected to a standardization process in step 296. The standardized concentrated slurry is then dried using a bench-top spray dryer in step 298 to result in a powder with an accurately determined concentration by weight of psychoactive alkaloids.

### 100% Water Solvent

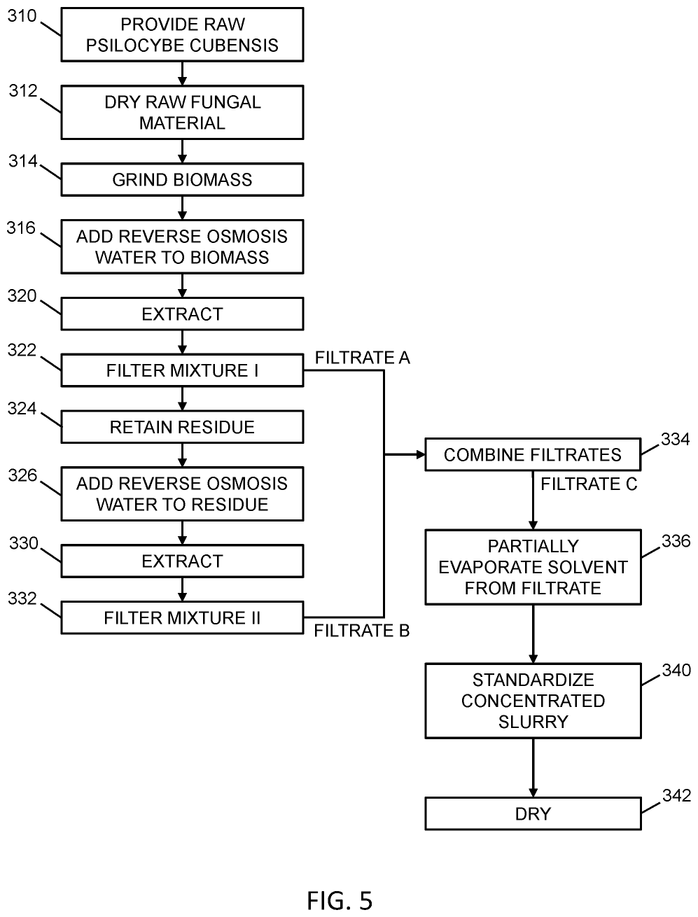

Referring to FIG. 5, a detailed process is shown for the extraction of psychoactive compounds Psilocybe cubensis using 100% reverse osmosis water as the solvent.

In step 310, 2.5 kg of raw psilocybin mushrooms from the Psilocybe cubensis species is provided. In step 312, the raw Psilocybe cubensis is dried in a forced air oven at 25° C. for 10 hours. The dried biomass is 140 g. Note that the dried biomass is the same weight in different examples because the mushrooms were from the same starting batch. In step 314, the dried biomass is ground in a hammer mill or the equivalent, to a particle size of 200 mesh.

In step 316, 5 L of solvent, which is 100% reverse osmosis water, is placed in an extraction vessel with the dried biomass, which is heat-controlled and agitated.

In step 320, the extraction proceeds. The temperature of the extraction process is 90° C., and the duration of the extraction is 12 hours. In the step 322, the extraction slurry is filtered while still hot to remove residue with undissolved Psilocybe cubensis from the filtrate. The filtrate from this step is considered as filtrate A. In step 324, the residue is retained and placed back in the extraction vessel. In step 326, another 5 L of 100% reverse osmosis water is added to the residue. In step 330, the extraction process of the residue continues at a temperature of 90° C., for 10 hours. The temperature remains constant during the extraction process. In step 332, the second resulting mixture, of biomass solids and water with dissolved extract, is filtered while still hot to remove the residue of unwanted solid material. Filtrates A and B are then mixed in step 334 to result in bulk filtrate C.

The bulk filtrate C is then processed with a rotary evaporator in step 336 to remove solvent until the volume of filtrate C is 2.5 L. At this point, the reduced amount of filtrate C is a concentrated slurry, due to the precipitation of some of the psychoactive alkaloids.

In step 340, after some of the solvent has been removed using the rotary evaporator, the concentrated slurry is then standardized. The standardization process uses a titration procedure to determine the concentration of the psychoactive alkaloids in the concentrated slurry. The standardization procedure entails adjusting the concentration of the psychoactive alkaloids in the concentrated slurry to a desired dry target. In this example, 6.3 g of ascorbic acid, 2.5 g of SiO₂ and 63 g of maltodextrin are added to the concentrated slurry.

In step 342, after the standardization process, the standardized concentrated slurry is dried using a bench-top spray dryer. This results in 140 g of powdered psilocybin mushroom extract with a total alkaloid concentration of 0.50% by weight.

### 100% Methanol

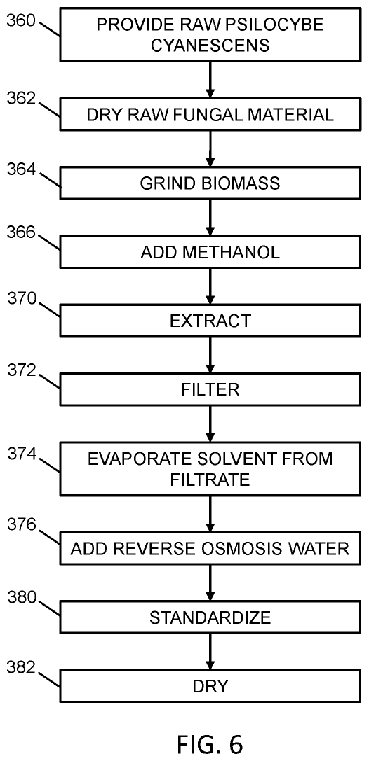

Referring to FIG. 6, a process is shown for the extraction of psychoactive compounds from Psilocybe cyanescens mushrooms using 100% methanol as the solvent.

In step 360, 2.5 kg of raw psilocybin mushrooms from the Psilocybe cyanescens species is provided. In step 362, the raw Psilocybe cyanescens is dried in a forced air oven at 25° C. for 10 hours. The dried biomass is 140 g. In step 364, the dried biomass is ground in a cutting mill or the equivalent, to particle size of 200 mesh. In step 366, 5 kg of solvent, which is 100% methanol, is added to an extraction vessel, which is heat-controlled and agitated. The dried biomass is also added to the extraction vessel.

In step 370, the extraction proceeds. The temperature of the extraction process is a constant 25° C., and the duration of the extraction is 4 hours. A pressure of 100 kPa above atmospheric (15 psig) is applied to the mixture of solvent and biomass during the extraction. In step 372, the extraction slurry is filtered to remove residue with undissolved Psilocybe cyanescens from the filtrate.

The filtrate is then processed with a rotary evaporator in step 374 to evaporate all the methanol from the filtrate. In this embodiment, all the solvent is removed at this stage because methanol is not regarded as safe for human consumption, and there should be no trace amounts of it remaining in the final product. In step 376, 1.25 L of reverse osmosis water at room temperature is added to the solid that is remaining after the evaporation step, to form a concentrated slurry.

In step 380, the concentrated slurry is standardized. In this example, 1.84 g of SiO₂ and 46 g of maltodextrin are added to the concentrated slurry. In step 382, the standardized concentrated slurry is dried using a bench-top spray dryer. This results in 95 g of powdered psilocybin mushroom extract with a total alkaloid concentration of 1.50% by weight.

### Acidic Solvent

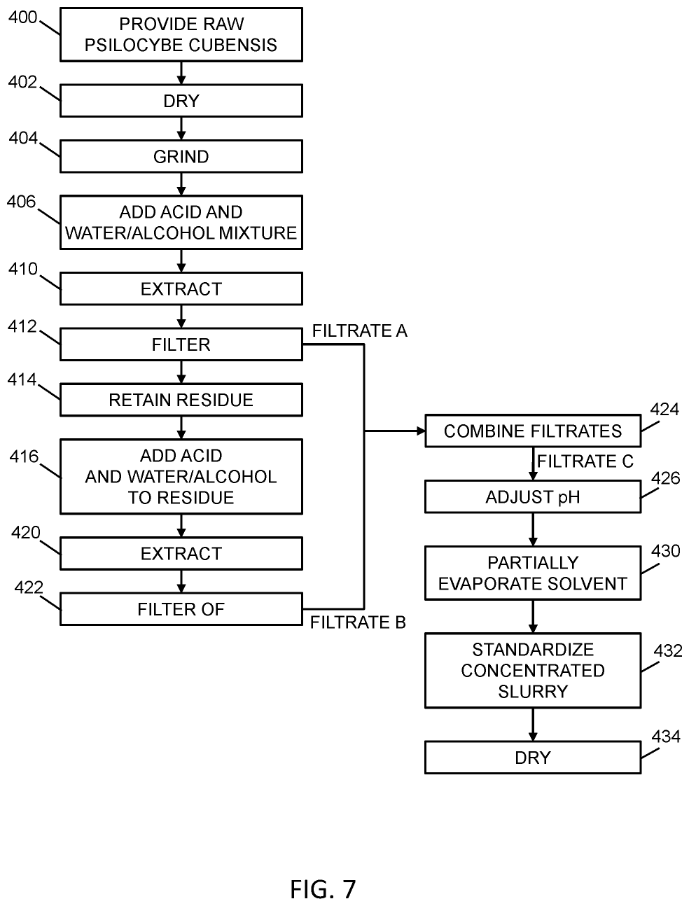

Referring to FIG. 7, a process is shown for the extraction of psychoactive compounds from Psilocybe cubensis mushrooms using a buffered acidic solvent. In step 400, 2.5 kg of raw psilocybin mushrooms from the Psilocybe cubensis species is provided. In step 402, the raw Psilocybe cubensis is dried in a forced air oven at 25° C. for 5-10 hours. The dried biomass is 140 g. In step 404, the dried biomass is ground in a hammer mill or the equivalent, to particle size of 200 mesh.

In step 406, 5 L of solvent is added with the dried biomass to an extraction vessel, which is heat-controlled and agitated. The solvent is a pH-adjusted, hydro-ethanol mixture. For its preparation, 44 g of anhydrous citric acid is placed into a 5 L vessel with 1.25 L of reverse osmosis water followed by 3.75 L of ethanol. The contents are mixed until completely dissolved. The pH of this solution is between pH 1.8 and pH 3. In some embodiments, the solvent is or is buffered with acetic acid, adipic acid, ascorbic acid, phosphoric acid, ammonium aluminum sulphate, ammonium citrate dibasic, ammonium citrate monobasic, calcium citrate, calcium fumarate, calcium gluconate, calcium phosphate dibasic, calcium phosphate, hydrochloric acid, sulphuric acid monobasic, calcium phosphate tribasic, citric acid, fumaric acid, gluconic acid, magnesium fumarate, malic acid, phosphoric acid, potassium acid tartrate, potassium citrate, potassium fumarate, sodium citrate, sodium fumarate, sodium gluconate, sodium lactate, sodium potassium hexametaphosphate, sodium potassium tartrate, sodium potassium tripolyphosphate, sodium pyrophosphate tetrabasic, sodium tripolyphosphate, tartaric acid, or any combination selected therefrom.

In step 410, the extraction proceeds. The temperature of the extraction process is 30° C., and the duration of the extraction is 4 hours. In step 412, the extraction slurry is filtered to remove residue with undissolved Psilocybe cubensis from the filtrate. The filtrate from this step is named filtrate A. In step 414, the residue is retained and placed back in the extraction vessel. In step 416, another 5 L of the same solvent is added to the residue. In step 420, the extraction process of the residue continues at a temperature of 30° C., for 4 hours. The temperature remains constant during the extraction process. In step 422, the second extraction slurry is filtered, to remove the residue of unwanted solid material. Filtrates A and B are then mixed in step 424 to result in bulk filtrate C.

Bulk filtrate C is then brought to a pH of 5 with 5M sodium hydroxide. The amount of the sodium hydroxide depends on the specific mushroom matrix extracted, and is not possible to predict accurately. The pH-adjusted, concentrated slurry is then processed with a rotary evaporator in step 430 to remove solvent until the volume of filtrate C is 2.5 L. At this point, the reduced amount of filtrate C is a concentrated slurry, due to the precipitation of some of the psychoactive alkaloids.

In step 432, the concentrated slurry is then standardized. In this example, 4.7 g of ascorbic acid, 1.9 g of SiO² and 47 g of maltodextrin are added to the concentrated slurry. In step 434, the standardized concentrated slurry is dried using a bench-top spray dryer. This results in 100 g of powdered psilocybin mushroom extract with a total alkaloid concentration of 1.00% by weight.

### Alkaline Solvent

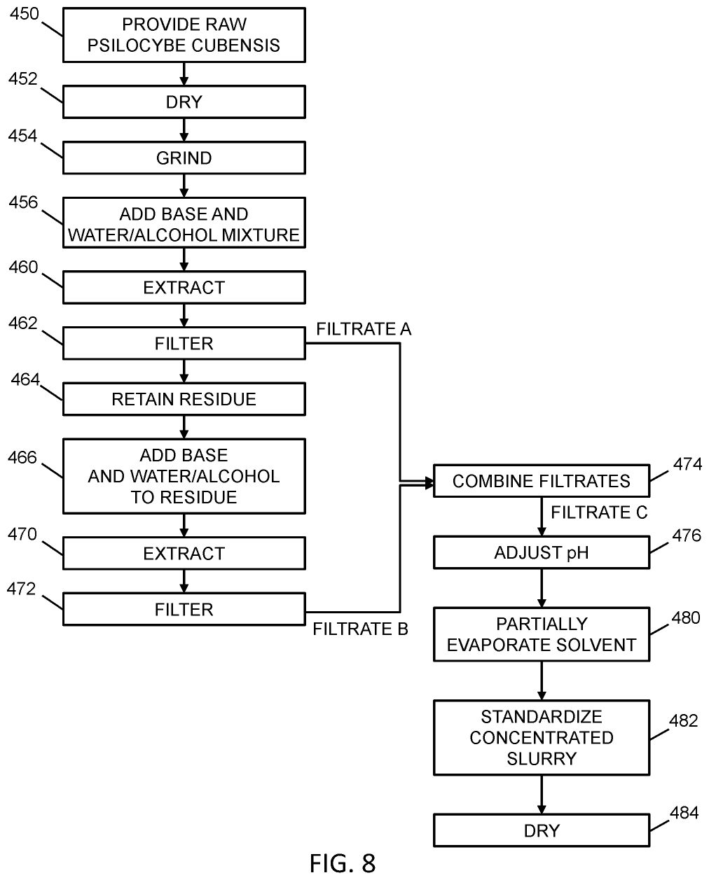

Referring to FIG. 8, a process is shown for the extraction of psychoactive compounds from Psilocybe cubensis mushrooms using a buffered alkaline solvent. In step 450, 2.5 kg of raw psilocybin mushrooms from the Psilocybe cubensis species is provided. In step 452, the raw Psilocybe cubensis is dried in a forced air oven at 25° C. for 10 hours. The dried biomass is 140 g. In step 454, the dried biomass is ground in a hammer mill or the equivalent, to a particle size of 200 mesh.

In step 456, 5 L of solvent is added with the biomass to an extraction vessel, which is heat-controlled and agitated. The solvent is a pH-adjusted, hydro-ethanol mixture. For its preparation, 200 g of sodium hydroxide pellets are placed into a 5 L vessel, with 1.25 L of reverse osmosis water followed by 3.75 L of ethanol. The contents are mixed until completely dissolved. The pH of this solution is between pH 11 and pH 12. In some embodiments, the solvent is buffered with ammonium bicarbonate, ammonium carbonate, ammonium hydroxide, calcium acetate, calcium carbonate, calcium chloride, calcium hydroxide, calcium lactate, calcium oxide, calcium phosphate, dibasic, calcium phosphate monobasic, magnesium carbonate, potassium aluminum sulphate, potassium bicarbonate, potassium carbonate, potassium hydroxide, potassium lactate, potassium phosphate, dibasic, potassium pyrophosphate, tetrabasic, potassium phosphate tribasic, potassium tripolyphosphate, sodium acetate, sodium acid pyrophosphate, sodium aluminum phosphate, sodium aluminum sulphate, sodium bicarbonate, sodium bisulphate, sodium carbonate, sodium hexametaphosphate, sodium hydroxide, sodium lactate, sodium phosphate dibasic, sodium phosphate monobasic, sodium phosphate tribasic, or any combination selected therefrom.

In step 460, the extraction proceeds. The temperature of the extraction process is 30° C., and the duration of the extraction is 4 hours. In step 462, the extraction slurry is filtered to remove residue with undissolved Psilocybe cubensis from the filtrate. The filtrate from this step is named filtrate A. In step 464, the residue is retained and placed back in the extraction vessel. In step 466, another 5 L of the same solvent is added to the residue. In step 470, the extraction process of the residue continues at a temperature of 30° C., for 4 hours. The temperature remains constant during the extraction process. In step 472, the second extraction slurry is filtered, to remove the residue of unwanted solid material. Filtrates A and B are then mixed in step 474 to result in bulk filtrate C.

Bulk filtrate C is then brought to a pH of 5 with sufficient 5M phosphoric acid. The pH-adjusted concentrated slurry is then processed with a rotary evaporator in step 480 to remove solvent until the volume of filtrate C is 2.5 L. At this point, the reduced amount of filtrate C is a concentrated slurry, due to the precipitation of some of the psychoactive alkaloids.

In step 482, the concentrated slurry is then standardized. In this example, 4.7 g of ascorbic acid, 1.9 g of SiO₂ and 47 g of maltodextrin are added to the concentrated slurry. In step 484, the standardized concentrated slurry is dried using a bench-top spray dryer. This results in 100 g of powdered psilocybin mushroom extract with a total alkaloid concentration of 1.00% by weight.

### D. Exemplary Embodiment for the Primary Process

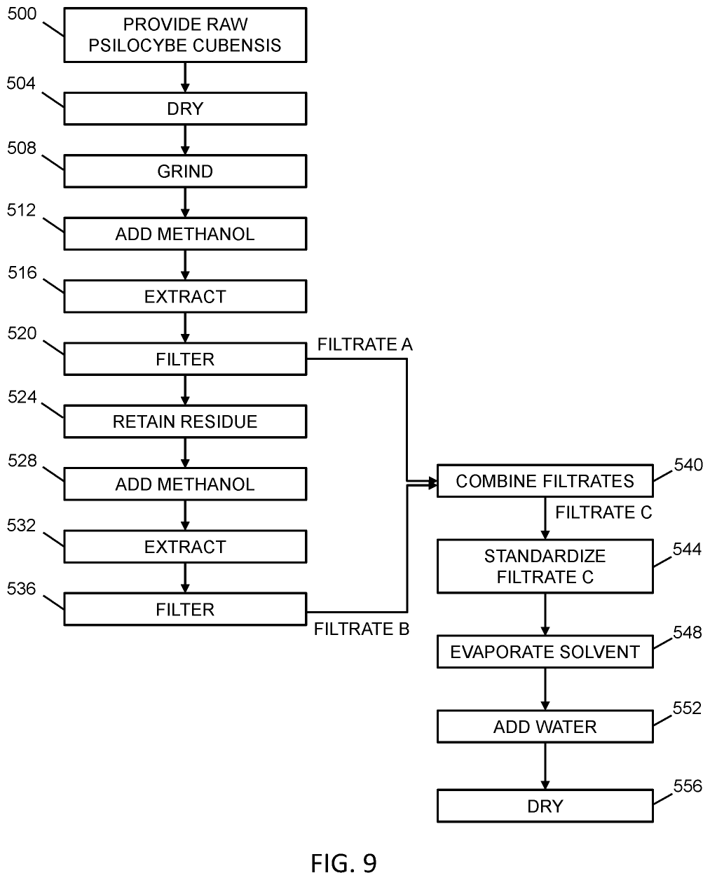

Referring to FIG. 9, a process is shown for the extraction of psychoactive compounds from Psilocybe cubensis mushrooms using 100% methanol as the solvent.

In step 500, a quantity of 2.5 kg of biomass of fresh, raw psilocybin fungus from the Psilocybe cubensis species is provided.

In step 504, the raw Psilocybe cubensis is dried in a forced air oven at 25° C. for 5-10 hours, resulting in 140 g of dried biomass.

In step 508, the dried biomass is ground or pulverized to a particle size of 200 mesh with a hammer mill to result in dried powdered biomass.

In step 512, the dried powdered biomass is placed into an agitated, heat-controlled vessel with 5.6 kg of methanol.

In step 516, the extraction proceeds. The temperature of the extraction process is a constant 25° C., and the duration of the extraction is 30 minutes. In some embodiments, pressure is applied during the extraction.

In step 520, the extraction slurry is filtered to remove residue with undissolved Psilocybe cubensis from the filtrate. The filtrate from this step 520 is filtrate A. In step 524, the residue is retained and placed back in the agitated, heat-controlled, extraction vessel. In step 528, another 5.6 kg of methanol by weight is added to the residue.

In step 532, the extraction process of the residue continues at a temperature of 25° C., for 30 minutes.

In step 536, the second extraction slurry is filtered through a 10 μm steel mesh filter, to remove the residue of unwanted solid material.

Filtrates A and B are then mixed in step 540 to result in a bulk filtrate C.

In step 544, the dry mass of the bulk filtrate C is analyzed and determined as well as the alkaloid concentration. The standardization process uses a titration procedure to determine the concentration of the psychoactive alkaloids in the bulk filtrate C. The standardization procedure entails adjusting the concentration of the psychoactive alkaloids in the bulk filtrate C, such that when it is dried the concentration reaches a desired dry target. The pooled extracts (bulk filtrate C) form 11,200 g of liquid extract at 0.5% solids containing 700 mg of total alkaloids (psilocybin and psilocin).

A quantity of 2.8 g of SiO₂, 0.140 g of ascorbic acid, and 81.06 g of maltodextrin are added to the bulk filtrate C in a mixing vessel and stirred, for example, until it is thoroughly mixed.

In step 548, the standardized bulk filtrate is immediately placed into a roto-evaporator and evaporated to dryness to evaporate all the methanol from the standardized bulk filtrate.

In step 552, the residue from the dried, standardized bulk filtrate is re-solubilized in water and mixed to make a 30% solids solution.

In step 556, the 30% solids solution is immediately subjected to a spray drying process using a bench-top spray dryer.

The final breakdown of the composition is: 56 g of extract (40%), 0.140 g of ascorbic acid (preservative, 0.1%), 2.80 g of SiO₂ (carrier 1, 2%), and 81.06 g of maltodextrin (carrier 2, 57.9%).

### E. Modifications to Use the Primary Process with Other Embodiments

Referring to FIG. 3, in order to use the primary process for this embodiment, the standardization of the concentrated slurry in step 260 is conducted before the partial evaporation of the solvent from the filtrate in step 256. Therefore, the filtrate C resulting from the combination step 254 is standardized before any of the 75% ethanol solvent from the resulting standardized filtrate is evaporated. The evaporation is then a complete or substantially complete evaporation instead of partial.

Referring to FIG. 4, in order to use the primary process for this embodiment, the standardization of the concentrated slurry in step 296 occurs before the partial evaporation of the solvent from the filtrate in step 294. Therefore, the filtrate resulting from the filtration step 292 is standardized before any of the solvent from the resulting standardized filtrate is evaporated. The evaporation is then a complete or substantially complete evaporation instead of partial.

Referring to FIG. 5, in order to use the primary process for this embodiment, the standardization of the concentrated slurry in step 340 is conducted before the partial evaporation of the water from the filtrate in step 336. Therefore, the filtrate C resulting from the combination step 334 is standardized before any of the water from the resulting standardized filtrate is evaporated. The evaporation is then a complete or substantially complete evaporation instead of partial.

Referring to FIG. 6, in order to use the primary process for this embodiment, the standardization of the concentrated slurry in step 380 occurs before the any of the evaporation of methanol from the filtrate in step 374. Therefore, the filtrate resulting from the filtration step 372 is standardized before any of the methanol from the resulting standardized filtrate is evaporated.

Referring to FIG. 7, in order to use the primary process for this embodiment, the standardization of the concentrated slurry in step 432 occurs before the partial evaporation of the acidic solvent from the filtrate in step 430. Therefore, the filtrate resulting from the combination step 424 and the pH adjustment step 426 is standardized before any of the acidic solvent from the resulting standardized filtrate is evaporated. The evaporation is then a complete or substantially complete evaporation instead of partial.

Referring to FIG. 8, in order to use the primary process for this embodiment, the standardization of the concentrated slurry in step 482 occurs before the partial evaporation of the alkaline solvent from the filtrate in step 480. Therefore, the filtrate resulting from the combination step 474 and the pH adjustment step 476 is standardized before any of the alkaline solvent from the resulting standardized filtrate is evaporated. The evaporation is then a complete or substantially complete evaporation instead of partial.

### F. Apparatus

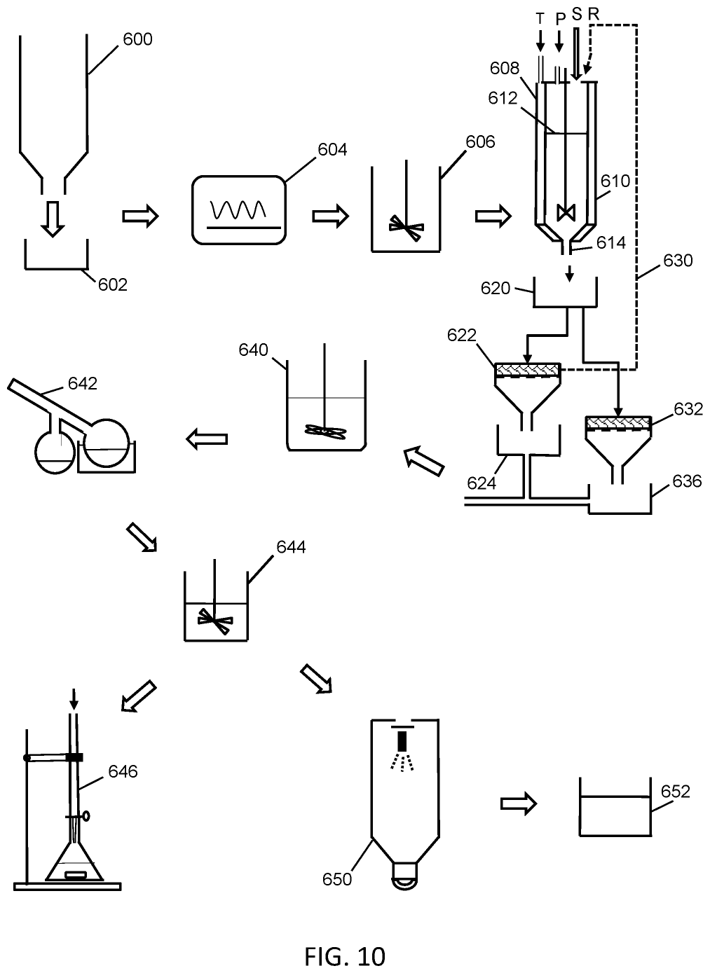

Referring to FIG. 10, an example of the apparatus is shown schematically. Raw psychedelic organism, such as psilocybin mushrooms are provided in a hopper 600, for example, and are released in batches into container 602. The raw psychedelic organism is then dried in a forced air oven 604. The dried biomass is placed into a grinder 606 for grinding.

After the drying and grinding steps, the ground biomass is placed in an agitated, heat-controlled extraction vessel 610. The vessel holds the biomass and solvent 612, such as lower aliphatic alcohols, C3-4 ketones, water, buffered acid or buffered alkaline, or any mixture of any thereof. The vessel may be surrounded by an insulating wall 608. Alternately, there may be an insulating jacket wrapped around the vessel. The insulating wall 608 or jacket helps to maintain the contents 612 under a constant temperature (T) between 5-95° C. The pressure (P) inside the extraction vessel 610 may be regulated up to 100 MPa (15000 psig).

After the extraction, the bottom of the extraction vessel 610 is opened at outlet 614 and the extraction slurry is collected in container 620. The extraction slurry is then fed into filter 622. After filtration, the first filtrate leaves the filter 622 and is collected in container 624. The residue 630 is then fed back at R into agitated, heat-controlled vessel 610 and more solvent (S) is added. After the second extraction, the extraction slurry is collected in container 620 and is then fed into filter 632 (or filter 622). After filtration, the second filtrate and solvent mixture leaves the filter 632 and is collected in container 636.

After the two filtration stages, if there are two, the filtrates are mixed in container 640. Otherwise, if there is only a single filtration step, mixing is unnecessary. Neutralizer is added as necessary to the filtrate in container 640.

Depending on the embodiment, the filtrate, pH-adjusted where necessary, is then passed to rotary evaporator 642 in which all or part of the solvent is evaporated. If all the solvent is evaporated, then reverse osmosis water is added to the solids remaining after the evaporation. The concentrated slurry is then passed to container 644 and tested to determine its alkaloid content, using a titration setup 646. Excipients are added to container 644 with the concentrated slurry, and mixed. The standardized slurry is then placed in a bench-top spray drier 650 to produce a psychedelic organism extract that is collected in container 652.

Instead, the filtrate may be tested in container 640 to determine its alkaloid content, using a titration setup 646. Excipients are added to container 640 with the filtrate, and mixed. The standardized filtrate is then passed to rotary evaporator 642 in which all or substantially all of the solvent is evaporated, depending on the embodiment. The resulting solids or slurry is then mixed with water in container 644 and placed in a bench-top spray drier 650 to produce a psychedelic organism extract that is collected in container 652.

### G. Variations

Other embodiments are also possible. While only specific neutralizing agents, food grade acids and food grade bases have been mentioned herein, other neutralizing agents, food grade acids and food grade bases may be used.

In general, unless otherwise indicated, singular elements may be in the plural and vice versa with no loss of generality.

Temperatures that have been given to the nearest degree include all temperatures within a range of ±0.5° C. of the given value. Likewise, numbers and percentages are specified to the nearest significant digit. Values of pH are specified to ±0.5.

While exemplary pH ranges are given in some examples, other pH ranges are possible.

Throughout the description, specific details have been set forth in order to provide a more thorough understanding of the invention. However, the invention may be practiced without these particulars. In other instances, well known elements have not been shown or described in detail and repetitions of steps and features have been omitted to avoid unnecessarily obscuring the invention. Accordingly, the specification and drawings are to be regarded in an illustrative, rather than a restrictive, sense. It will be clear to one having skill in the art that further variations to the specific details disclosed herein can be made, resulting in other embodiments that are within the scope of the invention disclosed. Steps in the flowchart may be performed in a different order, other steps may be added, or one or more may be removed without altering the main outcome of the process.

In other embodiments, other drying techniques, temperatures and durations may be used. It is possible in other embodiments to grind the dried biomass to lower or higher particle size than 200 mesh. For example, grinding to a mesh size of 40 would work in some embodiments. The choice of solvent may have an impact on which mesh size to grind the dried mushrooms to. Note that, in other embodiments, the grinding step 334 may take place before or after the drying step 332.

Water purified by other purification technologies may be used instead of reverse osmosis water. In alternative embodiments the solvent is 0.02% to 1.5% acetic acid in water. In alternate embodiment, the solvent comprises 75% ethanol, 25% water and 0.1M sodium hydroxide. In alternative embodiments the solvent is a hydro-methanol mixture, with a methanol content in the range of below 1% to 100%. The hydro-methanol based extraction follows the same steps as the extraction with a mixture of ethanol and water (FIG. 3), and may use lower soaking temperatures due to the lower boiling point of methanol. Also, the methanol/water mixture can be evaporated to dryness instead of the partial evaporation in step 294, for safety. If evaporated to dryness, the concentrated slurry is then formed by adding reverse osmosis water to the residual solid. If not evaporated to dryness, the residual slurry is diluted, if necessary for ease of handling, by adding reverse osmosis water to form the concentrated slurry. If not diluted, the residual slurry is used as the concentrated slurry. The result of evaporating the methanol is a residue that is either solid or a slurry. Furthermore, the hydro-methanol solvent may be buffered with a strong acid or a strong alkali, following the processes in FIGS. 6 and 7. Again, however, the solvent may be completely evaporated instead of partially (430, 480) in order to fully remove the methanol, with reverse osmosis water being added to the solid to form the concentrated slurry. If the solvent is not completely evaporated, it should be evaporated enough to remove all the methanol and leave a residual slurry. The residual slurry may optionally then be diluted, for ease of handling, with reverse osmosis water to form the concentrated slurry. If not diluted, the residual slurry is used as the concentrated slurry.

The solvent may also be propan-1-ol, propan-2-ol, a butanol isomer, or a mixture of any or all of these with water, in any percentage ratio.

Any of the solvents described herein may be used with any of the mushroom varieties that include psychoactive alkaloids.

The process may be scaled up using larger quantities and modified apparatus.

The extraction process in other embodiments may use varying applied pressures and temperatures, which vary during the soaking steps.

All parameters, dimensions, materials, quantities and configurations described herein are examples only and may be changed depending on the specific embodiment. Accordingly, the scope of the invention is to be construed in accordance with the substance defined by the following claims.

1. A process for forming an extract with a specified concentration of psychoactive alkaloids from a dried, raw psychedelic organism comprising the steps of:
soaking a biomass of dried, raw psychedelic organism with a solvent consisting of one or more members selected from the group consisting of C1-C4 aliphatic alcohols, C3-C4 ketones, water, a buffered acid and a buffered alkali in order to dissolve the psychoactive alkaloids in the solvent;
filtering an undissolved portion of the biomass from the solvent to result in a filtrate;
measuring a psychoactive alkaloid content in the filtrate;
measuring a dry mass content in the filtrate;
using the psychoactive alkaloid content, the dry mass content and the specified concentration to determine a quantity of excipient to add to the filtrate in order to obtain the specified concentration of the psychoactive alkaloids in the extract;
standardizing the filtrate by adding thereto the quantity of excipient; and
evaporating all or some of the solvent from the filtrate to leave the extract or a residue comprising the extract.

2. The process of claim 1, comprising:
mixing the extract or the residue in water to form a mixture; and
spray-drying the mixture to result in a powdered form of the extract.

3. The process of claim 1, wherein the solvent is methanol, a water-methanol mixture, acid buffered methanol, alkali buffered methanol, an acid buffered water-methanol mixture or an alkali buffered water-methanol mixture.

4. The process of claim 1, wherein the solvent is ethanol, a water-ethanol mixture, acid buffered ethanol, alkali buffered ethanol, an acid buffered water-ethanol mixture or an alkali buffered water-ethanol mixture.

5. The process of claim 1, wherein the solvent has a pH of 11-12, the process, comprising bringing the filtrate to a pH of 4-9.

6. The process of claim 1, wherein the solvent has a pH of 1.8-3, the process comprising bringing the filtrate to a pH of 4-9.

7. The process of claim 3, wherein the solvent is methanol.

8. The process of claim 7, wherein the solvent is fully evaporated from the filtrate.

9. The process of claim 1, wherein the soaking is at a temperature of 5-95° C.

10. The process of claim 1, comprising applying a pressure of 50 kPa-100 MPa to the solvent during the soaking step.

11. The process of claim 1, comprising agitating the solvent during the soaking step, wherein the soaking step has a duration of 10 minutes to 12 hours.

12. The process of claim 1, wherein the psychedelic organism is Amanita muscaria, Psilocybe cubensis, Psilocybe cyanescens, or any combination selected therefrom.

13. The process of claim 1, wherein the psychoactive alkaloids comprise psilocybin, psilocin, baeocystin, norbaeocystin, ibotenic acid or any combination selected therefrom.

14. The process of claim 1, wherein a ratio of the solvent to the biomass is in a range of 1 L: 1 kg to 50 L:1 kg.

15. The process of claim 1, wherein the specified concentration is 0.1-10%.

16. The process of claim 1, wherein the specified concentration is specified as a percentage with a precision of two decimal places.

17. The process of claim 1, wherein the excipient comprises ascorbic acid, silicon dioxide, maltodextrin, gum arabic, microcrystalline cellulose, sodium citrate, sodium benzoate, sodium phosphate, rice, rice hulls, or any combination selected therefrom.

18. The process of claim 1 comprising:
repeating, using further solvent, the soaking and filtering steps for the undissolved portion of the biomass to result in a further filtrate; and
combining the filtrate, after the filtering step, with the further filtrate.
